(19) United States
(12) Patent Application Publication
(10) Pub. No.: US 2019/0021339 A1
(43) Pub. Date: Jan. 24, 2019

(54) BIO-STIMULANT AND METHOD OF PRODUCING SAME

(71) Applicant: Acterra Tech Ltd., Vernon (CA)

(72) Inventor: Ralph Jeffery Lett, Vernon (CA)

(21) Appl. No.: 16/040,048

(22) Filed: Jul. 19, 2018

Related U.S. Application Data
(60) Provisional application No. 62/635,984, filed on Feb. 27, 2018.
(60) Provisional application No. 62/535,109, filed on Jul. 20, 2017.

(30) Foreign Application Priority Data
Jul. 20, 2017 (CA) 2973961
Feb. 27, 2018 (CA) 2996590

(51) Int. Cl. A01N 63/02; A01N 63/04; C05F 1/00; C05F 17/00

(52) U.S. Cl. CPC A01N 63/02; A01N 63/04; C12N 1/20; C05F 17/0036; C05F 1/00

(57) ABSTRACT

A method of producing an environmental bio-stimulant, the method comprising: preparing an aqueous mixture comprising a starting material including at least one microorganism, a first carbohydrate and water; placing the aqueous mixture in a microorganism growth environment for a first time interval; adding a second carbohydrate to the mixture; placing the mixture in a fermentation environment for a second time interval so as to produce the bio-stimulant. The method may include the further step of adding a carrier to the bio-stimulant. The carrier may be chosen from a group comprising water, zeolite, biochar, woodchips, or diatomaceous earth. The one or more microorganisms include a diverse plurality of groups of microorganisms.

### CROSS REFERENCE TO RELATED APPLICATIONS

This application claims priority from U.S. Provisional Application No. 62/535,109, filed Jul. 20, 2017, entitled: “Soil Inoculant and Method of Producing Same” and Canadian Patent Application No. 2,973,961 filed July 20, 2017, entitled: “Soil Inoculant and Method of Producing Same” and U.S. Provisional Application No. 62/635,984, filed Feb. 27, 2018, entitled “Bio-Stimulant and Method of Producing Same” and Canadian Patent Application No. 2,996,590 filed Feb. 27, 2018, entitled “Bio-Stimulant and Method of Producing Same”, entireties of which are incorporated herein by reference.

### FIELD

The present disclosure relates to a bio-stimulant product for inoculating soil or other environment and methods for producing same; in particular, the present disclosure relates to bio-stimulant products for introducing a diverse plurality of a group of beneficial microorganisms to a soil or other environment.

### BACKGROUND

In cultivating plants or trees, it is known that soil additives, commonly referred to as soil inoculants or bio-stimulants may be used to introduce beneficial microorganisms to the soil or growing medium of the plants, thereby promoting the growth and health of the plants being cultivated in that soil or growing medium. Plant health and growth is dependent on healthy soil biology. Beneficial microorganisms live in a symbiotic relationship with plants, supplying moisture and nutrients from the soil, and forming a natural defence system around the roots of plants, in exchange for glucose. For example, a class of fungi known as mycorrhizal fungi, effectively extend the surface area of the plant's root systems, thereby increasing the absorption of water and nutrients from the soil. A further example of a beneficial microorganism that exists in symbiosis with plants is a root colonizing species of bacteria known as rhizobacteria. Rhizobacteria inhabit the roots of plants, blocking disease causing bacteria from infecting the plant.

Although beneficial microorganisms may naturally be present in soil, the use of synthetic fertilizers has led to few beneficial microorganisms remaining in soil. Thus, it may be desirable to amend soil so as to introduce beneficial microorganisms, for example by using a bio-stimulant containing a diverse plurality of groups of beneficial microorganisms, comprising of bacteria, yeast and mold (fungi). Various different types of soil inoculants and bio-stimulants are available in the market, produced by various processes, some of which include first cultivating microorganisms on a host plant, and then harvesting the microorganisms from the host plant and processing it to thereby incorporate it into a soil inoculant product. For example, in patent application number PCT/ZA2008/000060 by inventor Venter (the '060 patent application), a method of producing endomycorrhiza inoculum involves cultivating endomycorrhiza spores on roots of a host plant in a growing medium and sequences of steps to separate the spores and producing a spore concentrate on a carrier. Specifically, the process embodiments described in the '060 patent application include removing a host plant from a growth medium and processing the growth medium containing spores and hyphae by washing it thoroughly with water or aqueous liquid, separating any root matter and coarse debris from the spore-containing liquid, separating the spores from the liquid, and mixing the spores with a fine absorbent carrier powder to obtain a nearly dry powder mixture. The carrier powder may include a fine zeolite powder and/or one or more clay minerals.

In patent application number PCT/ZA02/00046 by inventors Hilditch et al (the '046 patent application), a method of preparing a growth substrate for use in plant inoculation includes the steps of obtaining a viable source of microorganism, growing the microorganism in proximity to one or more propagative host plant root systems so as to encourage development of a symbiotic association between the microorganism and the propagative host plant root systems, supporting the microorganism and the propagative host plant root systems in a support medium being a suitable zeolite such as capezeo, allowing the microorganism to sporulate, terminating the growth of the propagative host plant root systems, allowing the microorganism tempter state of dormancy in response to the termination of growth of the propagative host plant root systems, and separating the microorganism, associated propagative host plant root systems and supports medium from the remainder of the propagative host plant.

In U.S. Pat. No. 8,728,460 by inventor Spittle (the '460 patent), a soil treatment composition is described which includes combining beneficial soil fungi and bacteria in a growth promoting nutrient medium and embedding it in an organic porous ceramic particle for direct delivery during soil aerification. The process of manufacturing the soil treatment composition, as described in the '460 patent, includes spraying the porous carrier particles with a biological soil treatment composition. Carbohydrates and other food sources for the dormant bacteria and fungi are included to increase the colony forming of the organisms.

In European patent application number 93913523.2 by inventor Sakai et al (the '523 patent application), a process for producing a substance inoculated with a vesicular arbuscular mycorrhizae (VAM) fungus is disclosed, which comprises cultivating a plant infected with a VAM fungus belonging to the genus Gigaspora by using a base material comprising calcined amber loam, or a mixture of calcined amber loam and calcined attapulgite, to thereby proliferate the VAM fungus. The process prepares a substance inoculated with a VAM fungus. Examples of carriers may include, for example, zeolite, foam clay, talc, pearlite, vermiculite, calcined amber loam, pumice, limestone, soil, sand, coke and peat moss. So as to prevent contamination of the carrier with indigenous microbes, the carrier is subjected to sterilization (including calcination) prior to its use. A sterilized soil or a calcined amber loam is preferably used. After the spore density has come to be sufficiently high, the use calcined amber loam or other carrier is recovered to isolate the formed VAM inoculant, which is optionally dried if desired to obtain the final inoculant product. In the examples given in the '523 patent application, the host plants are grown for a period of 16 weeks after transplantation before the soil inoculants were produced from the used calcined amber loam or other carriers.

### SUMMARY

The present disclosure relates to a bio-stimulant product and a method for producing same. The bio-stimulant product may be used to add beneficial microorganisms, such as bacteria, yeast, and fungal spores or fungi, and/or micronutrients, to soil, seed, compost, top dress, or other growth medium. The bio-stimulant product itself, produced by any of the processes disclosed herein, has beneficial advantages, and is itself intended to fall within the scope of the present disclosure. The bio-stimulant product functions as a biological pesticide and biological growth enhancer.

In one embodiment of the present disclosure, a method for producing a bio-stimulant product containing a diverse plurality of groups of microorganisms as well as other micronutrients includes using a starting material having sufficient amounts of beneficial microorganisms and, optionally, a desired mixture of micronutrients. The starting material may be, without intending to be limiting, humus, or soil from a thriving plant. The starting material is mixed together with water and an appropriate carbohydrate, for example a starchy carbohydrate, so as to fuel and facilitate the growth of the microorganisms. The aqueous starting material/carbohydrate mixture is left in the dark for approximately one week. A second carbohydrate, for example, sugar or molasses is then added to the mixture and the mixture is again placed in the dark for another two to four weeks allowing it to ferment, which turns the mixture into a water soluble, viscous mixture. A carrier, such as zeolite, biochar, woodchips, or diatomaceous earth may then be added, causing the diverse plurality of groups of microorganisms, as well as any available micronutrients, to become absorbed and/or adsorbed into the pores of the carrier and then the carrier is separated from the aqueous mixture and dried, preferably in the dark. Water may also be used as a carrier, eliminating the drying the carrier from not absorbed bio-stimulant.

In another aspect of the present disclosure, customized bio-stimulant products may be produced for particular applications. For example, certain species of beneficial bacteria and fungi may be particularly beneficial to certain types of plants, and so a bio-stimulant product may be customized so as to provide those particular bacterial and fungal species, in some embodiments also producing a particular ratio of those species, as well as customizing the particular types of micronutrients in accordance with the particular plant to be grown in the inoculated growth medium or soil. The bio-stimulant product may further be customized to have either an acidic, basic, or neutral pH by varying the carrier used, achieving the optimal pH environment for the plant to be grown using the bio-stimulant. For example, once inoculated, zeolite and diatomaceous earth are neutral, water and woodchips are acidic, and biochar is alkaline. If an alkaline pH environment is optimal for the plant's health and growth, biochar would be the most suitable carrier.

In further aspects of the present disclosure, the bio-stimulant product may be used to boost the immune system of animals, by increasing the number of beneficial microorganisms inhabiting the animal's skin, fur, feathers, or intestine. Further, the bio-stimulant product may be used to clean a production facility, for example a barn.

In other aspects of the present disclosure, the bio-stimulant product may be used for the treatment of manure, transforming the waste into a useable fertilizer and agriculture product. The method of using the bio-stimulant product for the treatment of waste is provided, the method comprising: applying a bio-stimulant product to manure for a first time interval; running the bio-stimulant and manure mixture through an apparatus adapted to apply centrifugal force; wherein the bio-stimulant, manure mixture is separated into a solid and a liquid component. The solid component, containing potassium, phosphate, and nitrogen may be used as a fertilizer, while the liquid component, containing high concentrations of potassium may have many uses in agriculture.

In some embodiments of the present disclosure, a method of producing a bio-stimulant product is provided, the method comprising: preparing an aqueous mixture comprising a starting material including one or more microorganisms, a first carbohydrate and water; placing the mixture in a microorganism growth environment for a first time interval; adding a second carbohydrate to the mixture; placing the mixture in a fermentation environment for a second time interval so as to produce the bio-stimulant.

### DETAILED DESCRIPTION

The methods and processes disclosed herein provide for the preparation and customization of bio-stimulants containing a diverse plurality of a group of microorganisms for various different applications. Beneficial microorganisms promote the growing process and in addition play a role in disease suppression. In one aspect of the present disclosure, a bio-stimulant may be prepared containing one or more groups of microorganisms, for example, which promote the growth of plants and trees by forming in the soil a symbiotic relationship between the microorganisms and the plants or trees underneath the soil. The diverse group of microorganisms found in the bio-stimulant, such as photosynthesizing bacteria, lactic acid bacteria, and yeasts, allow the microorganisms to survive in the soil environment and work synergistically to suppress the presence of harmful microorganisms.

In a preferred embodiment of the present disclosure, the bio-stimulant product includes photosynthesizing bacteria and lactobacillus bacteria, fermenting fungi and yeast, which function to fix nitrogen, solubilize phosphorus, and exert biocontrol.

As is known, beneficial microorganisms promote the healthy growth and development of plants and trees in soil, or other growth media, and are naturally present in soils with high organic material content. (The terms soil and growth medium are used interchangeably in the present disclosure). However, in agricultural and other environments the soil may be depleted of naturally occurring beneficial microorganisms over time, for example, through the use of synthetic fertilizers, thereby requiring reintroduction of the beneficial microorganisms to the soil so as to promote healthy plant growth. The diverse combination of microorganisms in the bio-stimulant product helps restore a healthy balance of microorganisms in the soil. Furthermore, micronutrients are essential to the healthy growth and development of various plants and trees. Similarly, while such micronutrients are often found in the natural environment, they may be depleted in certain areas over time where the soil has been used many times for growing plants or trees, for example on agricultural plots or in residential areas, and also thereby may require the addition or re-introduction of such micronutrients into the soil being used for growing plants.

In other prior art processes for producing bio-stimulants containing beneficial microorganisms, of which the applicant is aware, such processes usually involve cultivating the microorganisms on a host plant, typically for a period of several weeks, and then harvesting the microorganisms from the host plant to produce the bio-stimulant. In the processes and methods disclosed herein, by identifying a starting material which contains a group of beneficial microorganisms and which is preferably void of contaminants, such as harmful bacteria, the applicant has discovered that there are processes by which beneficial microorganisms may be cultivated directly from the starting material, without having to use host plants to cultivate the microorganisms over a period of weeks, and without the use of bioreactors and the like. Advantageously, the processes disclosed herein may therefore cost less and take less time to produce the bio-stimulant with desired concentrations of beneficial microorganisms for use in soil microorganism reintroduction. Furthermore, the applicant has found that the processes disclosed herein require fewer steps, in comparison to other processes, because it is not necessary to first separate microorganisms from the host plants in order to produce the bio-stimulant. Advantageously, in some embodiments of the present disclosure, it may be the case that the beneficial microorganism concentration of the bio-stimulants produced in the present processes disclosed herein may exceed the concentrations produced using other methods. Furthermore, the beneficial microorganisms included in the process may be customizable for the plant to be grown, by choosing an appropriate starting material, as described in further detail below.

As mentioned, the processes disclosed herein for producing bio-stimulants includes identifying an appropriate starting material. Different plants are adapted to grow in different soil compositions, and growing them in the wrong type of soil can be harmful to their health and growth. Healthy soil is the basis for a healthy and strong plant. Thus, the starting material may be chosen based on the plant intended to be grown. For example, and without intending to be limiting, if the targeted plant is a blueberry bush, the starting material may be taken from the soil of a blueberry bush that is a healthy, high yielding plant. The soil from a healthy blueberry bush will contain the diverse plurality of a group of microorganisms and nutrients necessary for enhancing the growth of subsequent blueberry plants.

A further example of an appropriate starting material, for bio-stimulants designed to reintroduce beneficial microorganisms into the soil for promoting plant growth, may include compost or humus, which contains the desired level of living biological activity. Compost or humus may contain microorganisms such as, lactobacillus, phosphate solubilizing bacteria, photosynthesizing bacteria and fermenting fungi and yeast. Additionally, such starting materials may also include different mixtures of certain micronutrients which are desirable for promoting healthy plant growth. Without intending to be limiting, in some embodiments of the present disclosure, for example, when plants are being grown for food, it may be desirable to select starting materials which do not contain undesirable contaminants, such as harmful bacteria, so as to prevent such contaminants from entering the human supply chain.

Without intending to be limiting, the applicant has sourced fungal dominated forest humus which has tremendous beneficial microbial diversity, and also includes various different micronutrients including in particular calcium, magnesium, copper, zinc, manganese and iron in quantities which are optimal for promoting healthy plant growth. The term “fungal dominated” as used herein means greater colony forming units of fungi than bacteria. The fungi found in fungal dominated forest humus aid in supressing pathogenic organisms and in creating healthy soil biology. In addition, the fungal dominated forest humus starting material includes smaller quantities of available nitrogen, phosphorus, potassium, and boron which again support the healthy growth of plants. Importantly, the forest humus source identified by the applicant for producing the bio-stimulant product does not contain harmful bacteria or plant disease pathogens, according to analyses conducted on the starting material.

While the above description of the specific starting material, comprising fungal dominated forest humus identified by the applicant, is an example of an appropriate starting material for the bio-stimulant production disclosed herein, it will be appreciated by a person skilled in the art that other appropriate starting materials may not contain these exact components, may include other components, and are intended to be included in the scope of the present disclosure. For example, other appropriate starting materials may contain different mixtures of microorganisms and micronutrients, or may not have any micronutrients or negligible amounts of micronutrients. Other examples of compounds or substances in a starting material that may be useful include fluvic and humic acid, which may increase the absorption of the microorganisms by the carrier, in the processes described below.

Once an appropriate starting material has been identified, the process for producing a bio-stimulant, in one aspect of the present disclosure, includes preparing an aqueous mixture comprising the identified starting material, a first carbohydrate, and water. Carbohydrates are a fuel source for microorganisms and play an important role in the growth and multiplication of microorganisms. Adding carbohydrates to the starting material is an important step for promoting the growth of the beneficial microorganisms. Without intending to be limiting, the first carbohydrate may be at least one starch. The prepared aqueous mixture is placed into a microorganism growth environment for a given time interval so as to facilitate the growth and multiplication of the microorganisms which exist in the starting material. For example, for producing a bio-stimulant which includes rhizobacteria and mycorrhizal fungi spores, the aqueous mixture would include a starting material which has rhizobacteria and mycorrhizal fungi. Without intending to be limiting, an example for the ratio of components in the aqueous mixture would be approximately 80% starting material, 10% water and 10% carbohydrate by volume.

As mentioned above, carbohydrates fuel the growth of the microorganisms. Without intending to be limiting, an appropriate carbohydrate may include, for example, oats, rice, but may include any other type of carbohydrate, including barley, grains, potato meal, cornstarch, coconut husks, peat, woodchips, corn or any other appropriate carbohydrate for promoting the growth of the targeted microorganisms, such as bacteria and fungi. Another carbohydrate source may include brewery waste, otherwise referred to as spent grain or leftover beer mash. Optionally, the carbohydrates may be ground up so as to increase the surface area of the carbohydrates in the aqueous mixture for the beneficial microorganisms to feed upon. In other embodiments in which a remediation product is produced, the food source for a remediation product containing nitrate-reducing bacteria may include chicken manure or other appropriate materials containing nitrate for the nitrate-reducing bacteria to feed upon.

Once the aqueous mixture has been prepared, it is placed in a microorganism growth environment which promotes the growth of the beneficial microorganisms that are desired to be produced. For example, the applicant has found that placing the aqueous mixture into a dark environment in which there is no light or air, combined with maintaining a temperature in that environment of approximately 30° C. for a first time interval of approximately one week effectively enables the beneficial microorganisms to multiply to the required concentrations for producing the bio-stimulant. However, it will be appreciated by a person skilled in the art that other microorganism growth environments may be appropriate for promoting the growth of beneficial microorganisms. For example, the temperature may vary in the range between 5° C. and 40° C., depending on the particular types of microorganisms being grown. In other embodiments, for example, for producing remedial products containing bacteria, the temperature range may be between 15° C. and 40° C., depending on the type of bacteria being grown.

The time interval for the microorganisms' growth need not be limited to seven days, and for example may be approximately in the range of four to fourteen days. The specific growing conditions for promoting microorganism growth may vary depending on the type of microorganisms which are being cultivated for the bio-stimulant or remediation product. The term “cultivate” as used herein means to grow. However, in the applicant's experience, in the embodiments for producing a bio-stimulant containing a diverse group of microorganisms, for example, comprising torulaspora delbruekii, acetobacter indonesiensis, acetobacter orientalis, acetobacter melorum, and sporolactobacillus nakayamae, or comprising lactobacillus mali, paenibacillus glycanilyticus, pichia membranifaciens, pichia manshurica, candida boidinii, lachancea fermentati, or comprising arthrobacter sp., candida membranifaciens, leuconostoc mesenteroides, penicillium canescens, geotrichum candidum, or comprising torulaspora delbruekii, bacillus subtilis, leuconostoc mesenteroides, brevibacterium frigoritolerans, or comprising pichia membranifaciens, wickerhamomyces anomalus, weissella paramesenteroids, bacillus megaterium, leuconostoc mesenteroides, raoultelle ornithinolytica, leaving the aqueous mixture in a lightless environment at a temperature of approximately 30° C. for approximately two to four weeks has produced sufficient microorganism growth for further processing to produce a bio-stimulant containing a diverse group of microorganisms.

Once the aqueous solution contains sufficient amounts of targeted beneficial microorganisms, a second carbohydrate, for example, at least one sugar, is added to the aqueous mixture, and then the aqueous mixture is placed into an environment adjusted for fermentation purposes for a second time interval. For example, without intending to be limiting, sugar may be introduced to the aqueous mixture at a ratio of one part sugar to two parts aqueous solution, by volume. The addition of sugar to the aqueous mixture enables fermentation of the mixture. The applicant has found that this process produces a substantially homogenous, viscous mixture, without chunks of humus or other starting material. Any type of sugar may be used for the fermentation process and is intended to be included in the scope of the present disclosure. Without intending to be limiting, different types of sugars which may be added include cane sugar, beet sugar, molasses, or other appropriate types of sugar for encouraging fermentation. The fermentation promoting environment may include, for example, placing the aqueous mixture with the sugar added into a dark environment in which there is no light or air, and maintaining the temperature of the environment in the range of 5° C. to 40° C., and preferably, in the range of approximately 30° C., for embodiments to produce bio-stimulants containing a diverse group of microorganisms comprising, for example, torulaspora delbruekii, acetobacter indonesiensis, acetobacter orientalis, acetobacter melorum, and sporolactobacillus nakayamae, or lactobacillus mall, paenibacillus glycanilyticus, pichia membranifaciens, pichia manshurica, candida boidinii, lachancea fermentati, or comprising arthrobacter sp., candida membranifaciens, leuconostoc mesenteroides, penicillium canescens, geotrichurn candidum, or torulaspora delbruekii, bacillus subtilis, leuconostoc mesenteroides, brevibacterium frigoritolerans, or pichia membranifaciens, wickerhamomyces anomalus, weissella paramesenteroids, bacillus megaterium, leuconostoc mesenteroides, raoultelle ornithinolytica. The second time interval, for example without intending to be limiting, may be approximately one to two weeks. In other embodiments for producing remedial products containing bacteria, the environment may be maintained at a temperature in the range of 15° C. to 40° C. The fermentation process also results in the production of volatile fatty acids, enzymes, and metabolites, which all play a role in disease suppression and are used by microorganisms to assist with reproduction and growth. In addition to facilitating fermentation, the applicant suspects the addition of sugar at this stage in the process may play a further role in extending the shelf life of the final bio-stimulant product, by providing a food source for the beneficial microorganisms. For example, without intending to be limiting, the shelf life of the final bio-stimulant product may be in the approximate range of one to two years. It will be appreciated by a person skilled in the art that the fermentation step described above, may be carried out more than once.

Once the fermentation is complete, the bio-stimulant is produced. Following fermentation, a carrier, such as water, zeolite, biochar, woodchips, or diatomaceous earth may be added to the bio-stimulant. It will be appreciated by a person skilled in the art that various types of material can be used as a carrier for the bio-stimulant product. Incorporation of the beneficial microorganism in a carrier enables easy handling and long term storage. Furthermore, the carrier can alter the pH of the soil. Soil pH can impact plant growth in several ways. Different microorganisms function best at different pH ranges. Soil pH may also impact the availability of micronutrients and minerals. By utilizing the appropriate carrier, the bio-stimulant can be customized to achieve an optimal pH growth environment for the targeted plant.

Once inoculated, zeolite and woodchips have an acidic pH and thus may be the carriers of choice when acidic conditions are optimal for plant health and growth. Water and diatomaceous earth, on the other hand, have a neutral pH. Biochar is alkaline and can be used to buffer acidity in soil, when an alkaline environment is optimal for the targeted plant. When zeolite, biochar, woodchips, or diatomaceous earth is chosen as the carrier, then a further step of drying the carrier may be carried out, so as to remove any access water from the carrier.

The applicant has found, in some aspects of the present disclosure, that the selection of an appropriate carrier will affect the final characteristics of the bio-stimulant product. For example, zeolites are alumina silica structures which provide a rigid crystalline network, whereby zeolite particles include pores and therefore have a large surface area compared to other types of crystalline structures. Biochar is a charcoal like product which is extremely porous in nature. Advantageously, both zeolites and biochar, having a large surface area are able to absorb and/or adsorb microorganisms, including fungal spores, as well as micronutrients, from the aqueous mixture. An additional benefit of zeolites and biochar is that they also absorb and/or adsorb water molecules into their pores and release the water molecules over time, which improves the water retention properties of the soil being treated with the bio-stimulant.

Depending on the particular environment to be remediated, such as a body of water, carriers other than zeolite and biochar, or in addition to zeolite or biochar, may be used in the production of a remediation product. For example, for remediating a body of water, a carrier which floats or which may be suspended in water may be selected. Examples of carriers other than zeolite may include different types of clays, talc powders, charcoals or a combination of any of these carriers.

During this stage of the process, the carrier is combined with the bio-stimulant at a ratio, for example, of approximately one part bio-stimulant, ten parts carrier and ten parts water by volume. Once the carrier has absorbed and/or adsorbed a sufficient amount of beneficial microorganisms and micronutrients, the carrier may be separated from the aqueous mixture, for example by filtering the carrier containing mixture through a sieve or screen. The recovered carrier is then dried so as to remove excess water and obtain the final bio-stimulant product. The drying process may occur, for example, in the dark (or in the absence of light in the UV spectrum), with dehumidifiers, for a period of time, which may take up to three days for example. Preferably, the drying process occurs in a temperature controlled environment, for example in the range of 15° C. It will be appreciated by a person skilled in the art that other drying procedures may be used and are intended to be included in the present scope of this disclosure. The aqueous mixture that is separated from the carrier may optionally be used as a liquid bio-stimulant product, as a certain amount of micronutrients and/or beneficial microorganisms remain within the aqueous solution.

In other aspects of the present disclosure, where it is desired to add micronutrients to the bio-stimulant, the careful selection of an appropriate starting material which contains those micronutrients may result in bio-stimulants including concentrations of those desired micronutrients. Therefore, in some embodiments of the present disclosure, it is not necessary to provide an additional source of micronutrients in the manufacture of the bio-stimulant because an appropriately selected starting material may already include those desired micronutrients. In other embodiments of the present disclosure, where the starting material does not contain the desired micronutrients or does not contain the desired concentration of micronutrients, such micronutrients may simply be introduced into the mixture during the manufacture process of the bio-stimulant, or may be included in the selected carrier to be added during the manufacture process. The addition of micronutrients may occur at any step in the process of producing the bio-stimulant.

In other aspects of the present disclosure, in addition to creating bio-stimulants for introducing beneficial microorganisms and optionally, micronutrients to the soil, other applications of the bio-stimulant product are available.

The bio-stimulant product may be applied to seeds as a seed inoculant. For example, without intending to be limiting, the bio-stimulant product, wherein the carrier is liquid, may be sprayed onto the seeds. The seeds may then, within 24 hours of being sprayed with the bio-stimulant product, be planted in the appropriate growth medium.

The bio-stimulant may be applied to manure or digestate to convert manure into fertilizer. For example, without intending to be limiting, manure or digestate may be soaked in the bio-stimulant product, wherein the carrier is liquid, for up to 2 weeks in large holding tanks. Then, the bio-stimulant and manure mixture may be pumped through a centrifuge, separating the mixture into a liquid and solid component. The solid component, made up of approximately 2 parts potassium, 2 parts phosphate and 2 parts nitrogen, may be used as a fertilizer. The liquid component, containing high concentrations of potassium, may have many uses in agriculture, as potassium plays a crucial role in a number of physiological processes vital to growth, health and yield of crops. Furthermore, application of the bio-stimulant product to the manure helps reduce odor.

Advantageously, the bio-stimulant product is a biological pesticide and may be used as a fungicide or bactericide. For example, mildew is a fungal disease affecting plants, characterized by a white coating on the surface of the affected parts of the plant. Mildew results in poor plant growth and lower yields. The applicant has found spraying affected plants with the bio-stimulant product wherein the carrier is liquid, and the product includes a plurality of beneficial microorganisms results in eradication of mildew. The method for producing the bio-stimulant product disclosed herein, has led to the discovery of a diverse plurality of groups of beneficial microorganisms that work synergistically to boost plant immunity and fight plant disease. A person skilled in the art would appreciate that the bio-stimulant product, wherein it contains a specific group of beneficial microorganisms, may be replicated in a bioreactor for use as a fungicide. For example, without intending to be limiting, for the treatment of mildew, the following group of beneficial microorganisms are successful at eradicating the disease: torulaspora delbruekii, acetobacter indonesiensis, acetobacter orientalis, acetobacter melorum, and sporolactobacillus nakayame.

The bio-stimulant product may also be used as a tool to promote animal health and growth by boosting animals' immune systems. Beneficial microorganisms found in the bio-stimulant product can help reduce or even eliminate disease-causing bacteria which may be found on an animal's skin, fur or feathers, or intestine, for example. When ingested, the bio-stimulant product may function as a probiotic, aiding in restoring the animal's natural balance of gut bacteria. The applicant has found that spraying livestock with the bio-stimulant product, wherein the carrier is liquid, results in weight gain without increasing the animals' diets. Furthermore, the applicant has found the use of the bio-stimulant product reduced the mortality rate of sick livestock, and overall resulted in healthier and stronger animals. The fungicidal and bactericidal properties of the bio-stimulant further allow the product to be used to clean production facilities, for example, a barn. The use of the bio-stimulant product results in accelerated composting of manure.

Other possible applications for the processes described herein include loading a carrier, such as zeolite or biochar, with other types of bacteria for remediating contaminated water, or for removing sour gas from sour gas wells or other types of wellheads. For example, without intending to be limiting, remediation products which contain oleispira antarctica bacteria may be used for consuming oil spills in cold water, alcaninvorax borkumensis bacteria for consuming oil spills in warm water, or nitrate-reducing bacteria which may consume hydrogen sulphide in sour gas wellheads or other types of wellheads. Without intending to be limiting, appropriate starting materials for producing a remediation product containing nitrate-reducing bacteria may include, for example, chicken manure. It will be appreciated by a person skilled in the art that various types of organic materials containing specific types of bacteria may be used as starting materials to produce bio-stimulant products or remediation products.

In a preferred embodiment of the present disclosure, the pH of the bio-stimulant product may be in the range of 3 to 4, as pathogenic microorganisms do not survive in acidic environments.

Wherein the bio-stimulant product is liquid (i.e. the carrier is liquid), it may preferably be stored in anaerobic, pressurized conditions, such as in a keg, or other similar pressurized container or vessel, with a one way valve. The applicant has found that the beneficial microorganisms survive longer in the anaerobic, pressurized environment, so as to increase the shelf life of the bio-stimulant product. Further, delivery from, for example, a keg, which maintains the controlled environment via the one way valve, and prevents air from contacting the bio-stimulant product, allows the product to be used on an as needed basis.

What is claimed is:

1. A method of producing a bio-stimulant, the method comprising the steps of:
a. preparing an aqueous mixture comprising a starting material including at least one microorganism; a first carbohydrate and water;
b. placing the aqueous mixture in a microorganism growth environment for a first time interval so as to cultivate the at least one microorganism;
c. adding a second carbohydrate to the mixture; and
d. fermenting the mixture in a fermentation environment for a second time interval so as to produce the bio-stimulant.

2. The method of claim 1 further comprising the step of adding a carrier to the bio-stimulant after fermentation.

3. The method of claim 2 wherein the carrier is selected from a group comprising: zeolite, biochar, woodchips, diatomaceous earth.

4. The method of claim 2 wherein the carrier is water.

5. The method of claim 3 further comprising the step of drying the carrier.

6. The method of claim 3, further comprising the step of using any of the bio-stimulant not carried by the carrier as a separate liquid bio-stimulant.

7. The method of claim 1 wherein the first carbohydrate is at least one starch.

8. The method of claim 1 wherein the second carbohydrate is at least one sugar.

9. The method of claim 1 wherein the first carbohydrate and second carbohydrate are the same.

10. The method of claim 7 wherein the at least one starch is selected from the group consisting of:
rice, oats, barley, grains, coconut husks, peat, woodchips, corn, and brewery waste.

11. The method of claim 8 wherein the at least one sugar is selected from the group consisting of:
cane sugar, brown sugar, molasses, and beet sugar.

12. The method of claim 1 wherein the starting material includes soil.

13. The method of claim 1 wherein the starting material includes humus.

14. The method of claim 13 wherein the humus is fungal dominated.

15. The method of claim 1 wherein the starting material includes micronutrients.

16. The method of claim 1 wherein the at least one microorganism includes a diverse plurality of groups of microorganisms.

17. The method of claim 16 wherein the diverse plurality of groups of microorganisms includes one or more bacteria and fungi.

18. The method of claim 17 wherein the bacteria is photosynthesising bacteria.

19. The method of claim 17 wherein the bacteria is lactobacillus.

20. The method of claim 17 wherein the fungi is fermenting fungi and yeast.

21. The method of claim 1 wherein the microorganism growth environment includes a lightless environment.

22. The method of claim 1 wherein the microorganism growth environment has a temperature in the range of substantially 5° C. to 40° C.

23. The method of claim 1 wherein the fermentation environment includes a lightless environment.

24. The method of claim 1 wherein the fermentation environment has a temperature in the range of substantially 5° C. to 40° C.

25. The method of claim 1 wherein the at least one microorganism includes at least one of:
torulaspora delbruekii, acetobacter indonesiensis, acetobacter orientalis, acetobacter melorum, and sporolactobacillus nakayamae, lactobacillus mall, paenibacillus glycanilyticus, pichia membranifaciens, pichia manshurica, candida boidinii, lachancea fermentati, arthrobacter sp., candida membranifaciens, penicillium canescens, geotrichurn candidum, bacillus subtilis, leuconostoc mesenteroides, brevibacterium frigoritolerans, wickerhamomyces anomalus, weissella paramesenteroids, bacillus megaterium, raoultelle ornithinolytica.

26. The method of claim 1 wherein the first time interval is selected from a range comprising substantially four days to seven days.

27. The method of claim 26 wherein the second time interval is selected from a range comprising substantially fourteen to thirty-two days.

28. A bio-stimulant produced by the method of claim 1.

29. The bio-stimulant of claim 28 wherein the bio-stimulant is stored in an anaerobic, pressurized, container having a one-way valve.

30. A method for the treatment of waste, the method comprising:
applying the bio-stimulant of claim 28 to manure for a first time interval;
running the bio-stimulant and manure mixture through an apparatus adapted to apply centrifugal force;
separating the bio-stimulant and manure mixture into a solid and liquid component.

31. The method of claim 30 wherein the first time interval is selected from a range comprising substantially seven days to fourteen days.
